(19) United States
(12) Patent Application Publication
(10) Pub. No.: US 2019/0279749 A1
(43) Pub. Date: Sep. 12, 2019

(54) PATIENT HEALTHCARE RECORD LINKING SYSTEM

(71) Applicant: KONINKLIJKE PHILIPS N.V., EINDHOVEN (NL)

(72) Inventors: Qingxin Wu, Lexington, MA (US); Reza Sharifi Sedeh, Malden, MA (US); Wei Wang, Somerville, MA (US); Yugang Jia, Winchester, MA (US)

(21) Appl. No.: 16/331,200

(22) PCT Filed: Aug. 30, 2017
(86) PCT No.: PCT/EP2017/071809

§ 371 Date: Mar. 7, 2019

Related U.S. Application Data
(60) Provisional application No. 62/385,560, filed on Sep. 9, 2016.

(51) Int. Cl. G16H 10/60; G06F 16/2457

(52) U.S. Cl. CPC G16H 10/60; G06F 16/24578

(57) ABSTRACT

The present disclosure pertains to a system configured to facilitate computer-assisted linkage of healthcare records. The system is configured to process a whole collection of records, using a reference set of record attributes, to generate a first prediction for a first portion of the collection of healthcare records of individuals of which healthcare records of the first collection portion have matching values with respect to the reference set of record attributes; re-process the first portion of records (the already matched portion of records) using other sets of record attributes to determine which ones of the other sets are reliable predictors of matching records; and use the reliable predictors to process a remaining unmatched portion of the healthcare records.

### BACKGROUND

### 1. Field

The present disclosure pertains to a system configured to facilitate computer-assisted linkage of healthcare records.

### 2. Description of the Related Art

Systems for computer-assisted linking of healthcare records are known. For example, healthcare records of an individual often exist in multiple data sources, one or more of which may include different record attributes, different formatting, or other differences. Missing data and/or data errors (e.g., typos) are also common in a large portion of records. For example, the social security number in a record for a patient may include the numbers “-01” in a Database A, but in Database B, the same social security number for the same patient has a typo and is stored as “-00”. Typical systems may not be able to match such records.

Because of the many potential differences between the records that can exist, the use of certain record attributes may be reliable for determining matches in one collection of records (e.g., with respect to accuracy of matches, sufficiency of matches, efficiency of determining matches, or other reliability criteria) but very unreliable for determining matches in another collection of records. When an entire collection of records (for which data linkage is desired) is processed for matches using record attributes that are unreliable for that record collection, an extensive amount of computational resources (e.g., processing resources, memory resources, network bandwidth, etc.) is likely exhausted but merely result in insufficient or inaccurate matches and record linkage.

### SUMMARY

Accordingly, one or more aspects of the present disclosure relate to a system configured to facilitate computer-assisted linkage of healthcare records. The system comprises one or more hardware processors and/or other components. The one or more hardware processors are configured by machine readable instructions to process, using a reference set of record attributes, a first portion of a collection of healthcare records of individuals to generate a first prediction of which healthcare records of the first collection portion have matching values with respect to the reference set of record attributes. The reference set of record attributes include one or more reference record attributes, and the first prediction indicates a first set of matches between healthcare records of the first collection portion. For each set of other sets of record attributes the one or more hardware processors are configured to: process, using the other set of record attributes, the first collection portion to generate a second prediction of which healthcare records of the first collection portion have matching values with respect to the other set of record attributes, each of the other sets of record attributes including one or more record attributes different from the one or more reference record attributes, and each of the second predictions indicating a second set of matches between healthcare records of the first collection portion; and determine, based on the first set of matches and the second set of matches, statistical information regarding use of the other set of record attributes for predicting healthcare record matches. The one or more hardware processors are further configured to select, based on the statistical information regarding use of one or more of the other sets of record attributes, at least one of the other sets of record attributes over at least another one of the other sets of healthcare record attributes for use in predicting healthcare record matches; and process, using the selected other set of record attributes, one or more other portions of the collection of healthcare records of individuals to generate a third prediction of which healthcare records of the other collection portions have matching values with respect to the selected other set of record attributes.

Yet another aspect of the present disclosure relates to a method for facilitating computer-assisted linkage of healthcare records with a linkage system. The system comprises one or more hardware processors and/or other components. The method comprises: processing, using a reference set of record attributes, a first portion of a collection of healthcare records of individuals to generate a first prediction of which healthcare records of the first collection portion have matching values with respect to the reference set of record attributes, the reference set of record attributes including one or more reference record attributes, and the first prediction indicating a first set of matches between healthcare records of the first collection portion. The method comprises, for each set of other sets of record attributes: processing, using the other set of record attributes, the first collection portion to generate a second prediction of which healthcare records of the first collection portion have matching values with respect to the other set of record attributes, each of the other sets of record attributes including one or more record attributes different from the one or more reference record attributes, and each of the second predictions indicating a second set of matches between healthcare records of the first collection portion; and determining, based on the first set of matches and the second set of matches, statistical information regarding use of the other set of record attributes for predicting healthcare record matches. The method comprises selecting, based on the statistical information regarding use of one or more of the other sets of record attributes, at least one of the other sets of record attributes over at least another one of the other sets of healthcare record attributes for use in predicting healthcare record matches; and processing, using the selected other set of record attributes, one or more other portions of the collection of healthcare records of individuals to generate a third prediction of which healthcare records of the other collection portions have matching values with respect to the selected other set of record attributes.

Still another aspect of the present disclosure relates to a system configured to facilitate computer-assisted linkage of healthcare records. The system comprises means for: processing, using a reference set of record attributes, a first portion of a collection of healthcare records of individuals to generate a first prediction of which healthcare records of the first collection portion have matching values with respect to the reference set of record attributes, the reference set of record attributes including one or more reference record attributes, and the first prediction indicating a first set of matches between healthcare records of the first collection portion; for each set of other sets of record attributes: processing, using the other set of record attributes, the first collection portion to generate a second prediction of which healthcare records of the first collection portion have matching values with respect to the other set of record attributes, each of the other sets of record attributes including one or more record attributes different from the one or more reference record attributes, and each of the second predictions indicating a second set of matches between healthcare records of the first collection portion; and determining, based on the first set of matches and the second set of matches, statistical information regarding use of the other set of record attributes for predicting healthcare record matches; selecting, based on the statistical information regarding use of one or more of the other sets of record attributes, at least one of the other sets of record attributes over at least another one of the other sets of healthcare record attributes for use in predicting healthcare record matches; and processing, using the selected other set of record attributes, one or more other portions of the collection of healthcare records of individuals to generate a third prediction of which healthcare records of the other collection portions have matching values with respect to the selected other set of record attributes.

These and other objects, features, and characteristics of the present disclosure, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the disclosure.

### BRIEF DESCRIPTION OF THE DRAWINGS

FIG. 1 is a schematic illustration of a system configured to facilitate computer-assisted linkage of healthcare records, in accordance with one or more embodiments.

FIG. 2 pictorially summarizes operations performed by the system, in accordance with one or more embodiments.

FIG. 3 is a flow chart that summarizes a portion (e.g., the portion after “Standardization” shown in FIG. 2) of the operations performed by the system, in accordance with one or more embodiments. FIG. 3 illustrates work flow of a decision model (e.g., a records matching algorithm).

FIG. 4 illustrates a method for facilitating computer-assisted linkage of healthcare records, in accordance with one or more embodiments.

### DETAILED DESCRIPTION OF EXEMPLARY EMBODIMENTS

As used herein, the singular form of “a”, “an”, and “the” include plural references unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other.

As used herein, the word “unitary” means a component is created as a single piece or unit. That is, a component that includes pieces that are created separately and then coupled together as a unit is not a “unitary” component or body. As employed herein, the statement that two or more parts or components “engage” one another shall mean that the parts exert a force against one another either directly or through one or more intermediate parts or components. As employed herein, the term “number” shall mean one or an integer greater than one (i.e., a plurality).

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

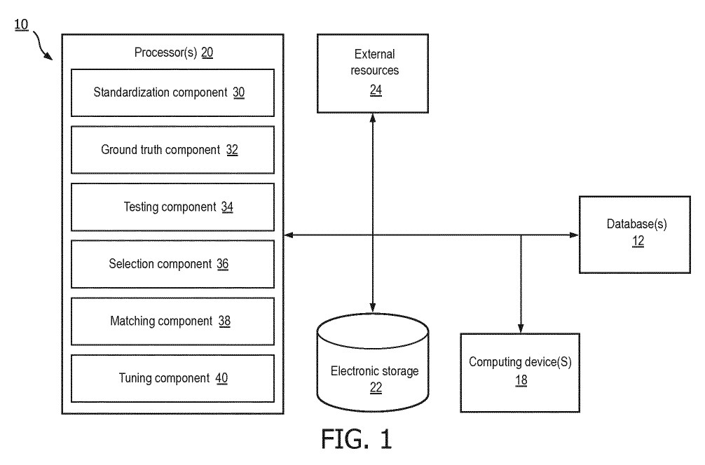

FIG. 1 illustrates a system 10 configured to facilitate computer-assisted linkage of healthcare records, in accordance with one or more embodiments. An individual healthcare patient may be associated with several different healthcare records stored in one or more different databases and/or other storage systems. For example, a single healthcare provider record system may include several different records for the same patient because the patient has used several different services offered by the healthcare provider. As another example, the patient may visit different doctors from different healthcare provider systems who each have their own records for the patient. Because of the private nature of healthcare information, healthcare records usually include record attributes (e.g., features) that identify individual patients. For example, the record attributes may include reference attributes (e.g., “strong” identifiers) such as social security number, name, and/or other attributes. Many records may be matched using values for these reference attributes alone. However, typos, missing entries (values), errors, and/or other record inconsistencies still render a considerable portion of records unmatchable with prior art systems. As such, use of certain record attributes traditionally used for matching records may not be reliable (e.g., with respect to accuracy of matches, sufficiency of matches, efficiency of determining matches, or other reliability criteria) for determining matches in one or more record collections that include records with the foregoing inconsistencies and/or records from different databases. Moreover, because of the many potential differences between the records that can exist, the use of certain record attributes may be reliable for determining matches in one collection of records but very unreliable for determining matches in another collection of records. When an entire collection of records (for which data linkage is desired) is processed for matches using record attributes that are unreliable for that record collection, an extensive amount of computational resources is likely exhausted but merely result in insufficient or inaccurate matches and record linkage. Although prior art systems may facilitate processing of such records to determine matches between the records and linking of the respective matching records by automating one or more operations to match and link records, typical prior art systems often exhaust an extensive amount of computational resources (e.g., processing resources, memory resources, network bandwidth, etc.) and produce inaccurate matches, insufficient matches, or other problematic issues (e.g., inefficient overall use of computational resources or other issues) before the unreliability of the record attributes used for the matching and linking of records is detected. Furthermore, as the number of records in a collection to processed increases (e.g., to hundreds of thousands of records, millions of records, billions of records, etc.), the negative effect caused by use of unreliable record attributes (e.g., for processing an entire collection of records) may exponentially grow, thereby furthering waste of computational resources.

In some embodiments, system 10 is configured to match a portion of records which include reference attributes that identify individual patients (e.g., “strong” identifiers). Using these known matched records, system 10 tests the reliability of other record attributes for matching the same records. System 10 then determines probabilistic matches between other records (e.g., records without “strong” identifiers and/or other records) based on the reliability evaluation of the other record attributes in the healthcare records. Advantageously, compared to prior art systems, system 10 may link healthcare records (including those without “strong” identifiers) with higher accuracy, greater number of matches, improved efficiency, or other benefits. In some embodiments, system 10 facilitates user customization of probability thresholds used for determining a degree to which records match (e.g., most matching systems return a binary result (match/not a match), which cannot be easily customized and does not indicate a degree to which records match), and system 10 does not require a pre-existing set of known matching records (e.g., manually annotated by users) for training a machine-learning algorithm to match records before such a system can be used.

In some embodiments, system 10 comprises one or more databases 12, one or more computing devices 18, one or more processors 20, electronic storage 22, external resources 24, and/or other components.

Database(s) 12 are configured to electronically store healthcare records of individuals and/or other information. The healthcare records may include a plurality of attributes (e.g., categories of information such as social security number, name, address, date of birth, doctor's name, treating facility, treatment description, treatment date, etc.) and corresponding values for the attributes (e.g., a social security number of 123-45-6789, a name of John P. Doe, 321 Main St., Jan. 1 1960, etc.). In some embodiments, corresponding attributes and values are attribute-value pairs. In some embodiments, the attribute-value pairs may be a name—value pair, key—value pair, field—value pair, and the like. The attributes include reference attributes (e.g., “strong” identifiers) and/or reference attribute combinations whose values and/or combinations of values uniquely identify individuals. For example, a social security number is enough, by itself, to identify an individual patient in a hospital record. Other examples of “strong” identifiers include a unique name, a phone number (e.g., including area code), a payer identification, and/or other identifiers.

Databases 12 are associated with one or more entities such as medical facilities (e.g., hospitals, doctor's offices, etc.), healthcare management providers (e.g., a veteran's affairs medical system, a ministry of health), health insurance providers, and/or other entities. Databases 12 comprise electronic storage media that electronically stores information. In some embodiments, databases 12 are and/or are included in computers, servers, and/or other data storage systems associated with the one or more entities. The electronic storage media of databases 12 may comprise system storage that is provided integrally (i.e., substantially non-removable) with such systems. Databases 12 may comprise one or more of optically readable storage media (e.g., optical disks, etc.), magnetically readable storage media (e.g., magnetic tape, magnetic hard drive, floppy drive, etc.), electrical charge-based storage media (e.g., EPROM, RAM, etc.), solid-state storage media (e.g., flash drive, etc.), and/or other electronically readable storage media. Databases 12 are configured to communicate with computing devices 18, processor 20, electronic storage 22, external resources 24, and/or other components of system 10 such that the information stored by databases 12 may be accessed (e.g., as described herein) by other components of system 10 and/or other systems. It should be noted that use of the term “databases” is not intended to be limiting. A database may be any electronic storage system that stores healthcare records and allows system 10 to function as described herein.

Computing devices 18 are configured to provide an interface between users and system 10. In some embodiments, computing devices 18 are associated with databases 12, processor 20 and/or a server that includes processor 20, a healthcare provider, individual users associated with the healthcare provider, service providers (e.g., consultants) to the healthcare provider, individual users of system 10, and/or other users and/or entities. Computing devices 18 are configured to provide information to and/or receive information from such users and/or entities. Computing devices 18 include a user interface and/or other components. The user interface may be and/or include a graphical user interface configured to present views and/or fields configured to receive entry and/or selection of healthcare records and/or information associated with healthcare records, present information related to matched healthcare records (e.g., matching probabilities, F-scores, record attributes), and/or provide and/or receive other information. In some embodiments, the user interface includes a plurality of separate interfaces associated with a plurality of computing devices 18, processors 20, and/or other components of system 10, for example.

In some embodiments, one or more computing devices 18 are configured to provide a user interface, processing capabilities, databases, and/or electronic storage to system 10. As such, computing devices 18 may include processors 20, electronic storage 22, external resources 24, and/or other components of system 10. In some embodiments, computing devices 18 are connected to a network (e.g., the internet). In some embodiments, computing devices 18 do not include processor 20, electronic storage 22, external resources 24, and/or other components of system 10, but instead communicate with these components via the network. The connection to the network may be wireless or wired. For example, processor 20 may be located in a remote server and may wirelessly receive healthcare records for matching from one or more healthcare providers. In some embodiments, computing devices 18 are laptops, desktop computers, smartphones, tablet computers, and/or other computing devices.

Examples of interface devices suitable for inclusion in the user interface include a touch screen, a keypad, touch sensitive and/or physical buttons, switches, a keyboard, knobs, levers, a display, speakers, a microphone, an indicator light, an audible alarm, a printer, and/or other interface devices. The present disclosure also contemplates that computing devices 18 include a removable storage interface. In this example, information may be loaded into computing devices 18 from removable storage (e.g., a smart card, a flash drive, a removable disk) that enables users to customize the implementation of computing devices 18. Other exemplary input devices and techniques adapted for use with computing devices 18 and/or the user interface include, but are not limited to, an RS-232 port, RF link, an IR link, a modem (telephone, cable, etc.) and/or other devices.

As shown in FIG. 1, processor 20 is configured via machine-readable instructions to execute one or more computer program components. The one or more computer program components may comprise one or more of a standardization component 30, a ground truth component 32, a testing component 34, a selection component 36, a matching component 38, a tuning component 40, and/or other components. Processor 20 may be configured to execute components 30, 32, 34, 36, 38, and/or 40 by software; hardware; firmware; some combination of software, hardware, and/or firmware; and/or other mechanisms for configuring processing capabilities on processor 20.

It should be appreciated that although components 30, 32, 34, 36, 38, and 40 are illustrated in FIG. 1 as being co-located within a single processing unit, in embodiments in which processor 20 comprises multiple processing units, one or more of components 30, 32, 34, 36, 38, and/or 40 may be located remotely from the other components. The description of the functionality provided by the different components 30, 32, 34, 36, 38, and/or 40 described below is for illustrative purposes, and is not intended to be limiting, as any of components 30, 32, 34, 36, 38, and/or 40 may provide more or less functionality than is described. For example, one or more of components 30, 32, 34, 36, 38, and/or 40 may be eliminated, and some or all of its functionality may be provided by other components 30, 32, 34, 36, 38, and/or 40. As another example, processor 20 may be configured to execute one or more additional components that may perform some or all of the functionality attributed below to one of components 30, 32, 34, 36, 38, and/or 40.

Standardization component 30 is configured to obtain and/or otherwise identify healthcare records for matching. Standardization component 30 is configured to obtain healthcare records from, and/or identify healthcare records in, one or more databases 12. For example, standardization component 30 may obtain a plurality of records for matching from a single database 12 and/or may obtain the plurality of records from a plurality of databases 12 (e.g., with one or more records obtained from the individual databases 12). Standardization component 30 is configured to standardize the information in the healthcare records for analysis by ground truth component 32, testing component 34, selection component 36, matching component 38, tuning component 40, and/or other components of system 10. Standardizing the information may include formatting the information in individual records in the same way, eliminating unneeded and/or extraneous information from individual records, identifying attributes and/or values in the records, and/or other standardization. In some embodiments, standardization operations performed by standardization component 30 may be different for different records. For example, records from a first database 12 may have a first format which may be reformatted to a standard format by standardization component 30. Records from a second database 12 (and/or the first database 12) may already be formatted with the standard format but may include extraneous information removed by standardization component 30. In some embodiments, values of same field in different databases may be different. For example, the value of gender can be ‘M’ and ‘F’, ‘Male’ and ‘Female’, ‘Man’ and ‘Woman’, and so on. Standardization component 30 may standardize inconsistencies like these and/or other inconsistencies before further processing (e.g., as described below) of information by system 10.

Ground truth component 32 is configured to predict, using a reference set of record attributes (e.g., features), matching records in a first portion of a collection of healthcare records. The reference set of record attributes is used to process the first portion of the collection of healthcare records of individuals to generate a first prediction of which healthcare records of the first collection portion have matching values with respect to the reference set of record attributes. The reference set of record attributes includes one or more reference record attributes (e.g., individual features and/or feature combinations). The reference set of record attributes comprises one or more record attributes and/or a combination of record attributes that are known to be reliable for accurately predicting a match between two healthcare records when the known reliable attributes or combination of record attributes of the two healthcare records have respective matching values. The first prediction indicates a first set of matches between healthcare records of the first collection portion. These matched records may be used as described below to determine the reliability of other record attributes for predicting matches between other healthcare records.

By way of a non-limiting example, ground truth component 32 may determine known reliable features and/or feature combinations for a first portion of patient healthcare records in a first database and match, using at least one of the known reliable features and/or feature combinations, the first portion of patient healthcare records in the first database to a first portion of corresponding patient healthcare records in a second database that share the same values for the at least one known reliable feature and/or feature combination.

Testing component 34 is configured to use other sets of record attributes to predict matching records in the first portion of the collection of healthcare records. For each set of other sets of record attributes the other set of record attributes is used to process the first collection portion to generate a second prediction of which healthcare records of the first collection portion have matching values with respect to the other set of record attributes. Each of the other sets of record attributes includes one or more record attributes different from the one or more reference record attributes, and each of the second predictions indicates a second set of matches between healthcare records of the first collection portion. In some embodiments, at least one of the other sets of record attributes includes no personally identifiable information attributes (e.g., the at least one of the other sets of record attributes does not include a social security number, a unique name, a phone number (including are code), etc.). For example, the matching prediction operation performed by ground truth component 32 may be rerun by testing component 34 on the same data (e.g., the first portion of the collection of healthcare records), but with different sets of record attributes to determine whether the different sets of record attributes predict the same record matches already known by way of the reference (e.g., the known reliable) attributes.

Testing component 34 is further configured to determine statistical information regarding the use of the other sets of record attributes for predicting healthcare record matches. The statistical information is determined based on the first set of matches and the second set of matches (e.g., how well do the second set of matches match the first set of matches), and/or other information. In some embodiments, testing component 34 may be testing the reliabilities of the other sets of record attributes as record matching predictors (e.g., do the other sets of record attributes predict the same matches predicted by the reference sets of attributes?). In some embodiments, the statistical information includes information regarding one or more true positives, false positives, true negatives, and/or false negatives related to predicted matches in the second set of matches relative to the first set of matches. In some embodiments, true negatives, for example, may not be included because of the potential for true negatives to dominate an analysis such that the other three values have little or no impact on the analysis. In some embodiments, the statistical information comprises F-scores and/or other information for individual other sets of record attributes.

Selection component 36 is configured to select at least one of the other sets of healthcare record attributes over at least another one of the other sets of healthcare record attributes for use in predicting healthcare record matches. The selection is made based on the statistical information and/or other information (e.g., based on the determined reliabilities of the other sets of healthcare record attributes). In some embodiments, selection component 36 is configured to compare F-scores and/or other information for the individual other sets of record attributes and select, for use in predicting healthcare record matches, at least one of the other sets of record attributes based on the comparison. In some embodiments, selection component 36 is configured to select at least one of the other sets of record attributes based on the comparison indicating the selected other set of record attributes has an F-score greater than or equal to an F-score for at least another one of the other sets of record attributes. For example, selection component 36 may be configured to rank the other sets of record attributes based on their F-scores. In some embodiments, selection component 36 is configured to select at least one of the other sets of record attributes based on the comparison indicating the selected other set of record attributes has an F- score that satisfies a reliability threshold. The reliability threshold may be determined at manufacture, determined and/or adjusted by a user via a computing device 18 associated with the user, and/or determined in other ways.

In some embodiments, ground truth component 32, testing component 34, and/or selection component 36 are configured such that the reference set of record attributes (described above) has a reference reliability score. The reference reliability score is based on accuracy of the first set of predictions. Selection component 36 may be configured to set the F-score (for example) reliability threshold for the other sets of record attributes based on the reference reliability score and/or other information. For example, the reliability threshold for the match predicting ability of the other sets of record attributes may greater than, greater than or equal to, or no less than the reference reliability record by a given percentage or amount.

Matching component 38 is configured to process one or more other portions of the collection of healthcare records of individuals, using the selected other set of record attributes, to generate a (e.g., third) prediction of which healthcare records of the other collection portions have matching values with respect to the selected other set of record attributes. Matching component 38 is configured to determine a matching probability (e.g., a percentage and/or other indicators of a likelihood of a match) for individually matched records. The matching probabilities for individually matched records are determined based on the statistical information determined by testing component 34, and/or other information. In some embodiments, the matching probabilities for the individually matched records are and/or correspond to (e.g., are a function of) the determined reliabilities of the selected other sets of record attributes (e.g., the F-scores) used to match the records. For example, if an F-score for a selected other set of record attributes used to match a particular set of records was 0.85, the matching probability determined by matching component 38 for that set of records may be some function of the F-score. An F-score itself may or may not be a good indicator of matching probability. As described above, an F-score is a value between 0 and 1, and a higher value has a positive correlation with matching probability. However, an F-score alone may not meet a user's requirements for an indicator of matching strength. Thus, a matching component 38 may be configured such that the determined matching probability is some function of the F-score such that an F-score is scaled to a final matching probability determination sufficient for a user.

In some embodiments, matching component 38 is configured to iteratively use a highest ranked (e.g., based on the F-scores and/or other indicators of reliability) other set of record attributes, a next highest ranked other set of record attributes, and so on, to generate predictions of which healthcare records of the other collection portions have matching values. This matching may continue within an iteration and/or across multiple iterations until stopping criteria is satisfied. In some embodiments, the stopping criteria comprises one or more of predicting matches for a predetermined quantity of records, a particular set of record attributes whose predicted matches have a matching probability that breaches a matching probability threshold level, a lack of remaining other sets of record attributes whose F-scores breach a reliability threshold, and/or other criteria. For example, matching component 38 may be configured to process, using a higher ranked (based on the F-score for example) first selected other set of record attributes, another portion of the collection of healthcare records of individuals to generate the (third) prediction until matching probabilities for the matches predicted by the first selected other set of record attributes drop below 80% (80% is used as a non-limiting example). Next, matching component 38 may process, using an F-score based next most reliable other set of record attributes, a further portion of the collection of healthcare records of individuals to generate a (e.g., fourth) prediction of which healthcare records of the further portion have matching values with respect to the next most reliable other set of record attributes until a predetermined number of matches is reached. It should be noted that this process may continue for more than the two iterations described in this example.

In some embodiments, matching component 38 is configured to facilitate adjustment of the reliability threshold for sets of record attributes, the stopping criteria, a matching probability threshold, and/or other features of system 10. Matching component 38 is configured to facilitate adjustment via the user interface of computing devices 18 and/or other by other methods. For example, matching component 38 may cause presentation of one or more views of the graphical user interface that include one or more fields for receiving entry and/or selection of threshold values, record matching quantities, and/or other information from a user.

In some embodiments, matching component 38 is configured to electronically link matched records. Electronically linking matched records may include establishing an electronic association between matched records. The electronic association may indicate a common patient and/or other entities to which the linked records refer. In some embodiments, the electronic link between matched records may facilitate storage of the linked records in a common electronic repository, electronic navigation from one linked record to another, physically obtaining copies of the linked records, and/or other operations.

Tuning component 40 is configured to adjust the matching probabilities for individual record matches determined by matching component 38. Tuning component 40 is configured to adjust the matching probabilities determined by matching component 38 based on edit distances associated with values of record attributes in the matched records and/or other information. For example, if system 10 matched two records with differing social security numbers based on other record attributes (e.g., features and/or feature combinations), tuning component 40 may determine an edit distance associated with the social security numbers (and/or other attributes) and tune the matching probability for the two records based on the edit distance. In this example, an edit distance of “1” may mean there is only a 1 digit difference between the two social security numbers, which, for example, may be a simple typo. In this case, tuning component 40 may increase the matching probability for these records (e.g., from 85% to 90%). However, if an edit distance was large (e.g., multiple differing digits in the social security number example possibly indicating a totally different social security number) tuning component may decrease the matching probability for these records. In some embodiments, tuning component 40 is configured such that the most a matching probability may be increased is an amount that increases the matching probability to a level that corresponds to the reliability (e.g., the F-score) of an immediately previous higher ranked set of record attributes, and the most a matching probability may be decreased is an amount that decreases the matching probability to a level that corresponds to the reliability (e.g., the F-score) of an immediately following lower ranked set of record attributes.

In some embodiments, system 10 may facilitate user review of the matched records. In some embodiments, system 10 may facilitate user review of matched records whose tuned matching probabilities are at or near the matching probability threshold level described above. In some embodiments, system 10 may facilitate user review of non-matched records. Facilitating review may include causing a computing device 18 associated with a user to present information related to the matched records to the user. The information related to the matched records may include, for example, the record attributes used to match the records, the values of the record attributes, the F-score for the record attributes, the tuned matching probability determined for the matched records, the records themselves, and/or other information. In some embodiments, a user may adjust one or more of the thresholds described herein and/or take other actions based on the user review.

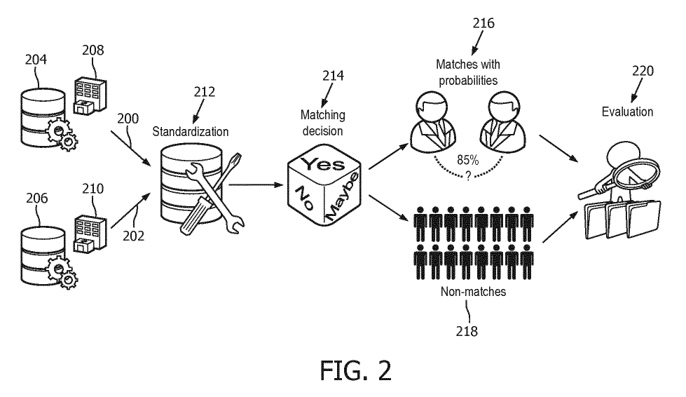

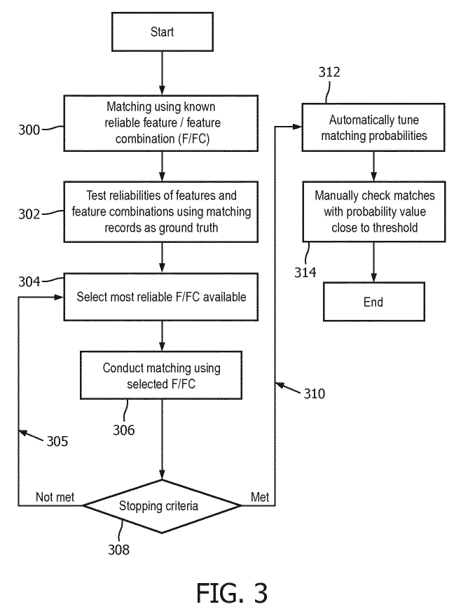

FIG. 2 and FIG. 3 summarize operation s performed by system 10 (shown in FIG. 1). FIG. 2 pictorially summarizes operations performed by system 10. FIG. 3 is a flow chart that summarizes operations performed by system 10. In the example shown in FIG. 2, system 10 obtains 200, 202 healthcare records from two different databases 204, 206 associated with two different entities 208, 210. The records are standardized 212 and matched 214 as described herein. Matching probabilities (e.g., 85%) are determined 216 for matched records. In some embodiments, non-matching records may also be identified 218. Finally, system 10 is configured to facilitate user review (evaluation) 220 of matched and/or non-matched records.

As shown in FIG. 3, system 10 (shown in FIG. 1) is configured to match a first portion of records using known reliable features and/or feature combinations (sets of record attributes. The matched first portion of records is used to test 302 reliabilities of other features and/or feature combinations (other sets of record attributes). The most reliable features and/or feature combinations (sets of record attributes) are selected 304 and used to iteratively 305 match 306 other records until stopping criteria 308 is met 310. System 10 automatically tunes 312 matching probabilities and facilitates 314 manual reviews of matched records.

Returning to FIG. 1, electronic storage 22 comprises electronic storage media that electronically stores information. The electronic storage media of electronic storage 22 may comprise one or both of system storage that is provided integrally (i.e., substantially non-removable) with system 10 and/or removable storage that is removably connectable to system 10 via, for example, a port (e.g., a USB port, a firewire port, etc.) or a drive (e.g., a disk drive, etc.). Electronic storage 22 may be (in whole or in part) a separate component within system 10, or electronic storage 22 may be provided (in whole or in part) integrally with one or more other components of system 10 (e.g., a computing device 18, processor 20, etc.). In some embodiments, electronic storage 22 may be located in a server together with processor 20, in a server that is part of external resources 24, in computing devices 18, and/or in other locations. Electronic storage 22 may comprise one or more of optically readable storage media (e.g., optical disks, etc.), magnetically readable storage media (e.g., magnetic tape, magnetic hard drive, floppy drive, etc.), electrical charge-based storage media (e.g., EPROM, RAM, etc.), solid-state storage media (e.g., flash drive, etc.), and/or other electronically readable storage media. Electronic storage 22 may store software algorithms, information obtained and/or determined by processor 20, information received via computing devices 18 and/or other external computing systems, information received from external resources 24, information received from database(s) 12, and/or other information that enables system 10 to function as described herein. By way of a non-limiting example, electronic storage 22 may store F-scores for the individual features and/or feature combinations.

External resources 24 include sources of information (e.g., databases, websites, etc.), external entities participating with system 10 (e.g., a medical records system of a health care facility), one or more servers outside of system 10, a network (e.g., the internet), electronic storage, equipment related to Wi-Fi technology, equipment related to Bluetooth® technology, data entry devices, and/or other resources. In some implementations, some or all of the functionality attributed herein to external resources 24 may be provided by resources included in system 10. External resources 24 may be configured to communicate with processor 20, computing device 18, electronic storage 22, database(s) 12, and/or other components of system 10 via wired and/or wireless connections, via a network (e.g., a local area network and/or the internet), via cellular technology, via Wi-Fi technology, and/or via other resources.

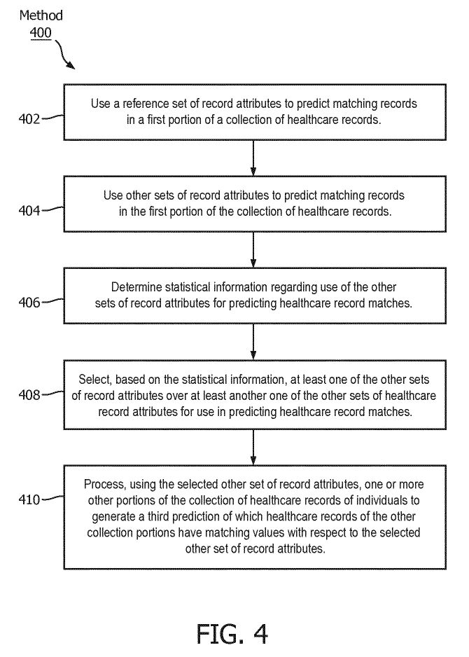

FIG. 4 illustrates a method 400 for facilitating computer-assisted linkage of healthcare records, in accordance with one or more embodiments. Method 400 may be performed with a linkage system. The system comprises one or more hardware processors and/or other components. The one or more hardware processors are configured by machine readable instructions to execute computer program components. The computer program components include a standardization component, a ground truth component, a testing component, a selection component, a matching component, a tuning component, and/or other components. The operations of method 400 presented below are intended to be illustrative. In some embodiments, method 400 may be accomplished with one or more additional operations not described, and/or without one or more of the operations discussed. Additionally, the order in which the operations of method 400 are illustrated in FIG. 4 and described below is not intended to be limiting.

In some embodiments, method 400 may be implemented in one or more processing devices (e.g., a digital processor, an analog processor, a digital circuit designed to process information, an analog circuit designed to process information, a state machine, and/or other mechanisms for electronically processing information). The one or more processing devices may include one or more devices executing some or all of the operations of method 400 in response to instructions stored electronically on an electronic storage medium. The one or more processing devices may include one or more devices configured through hardware, firmware, and/or software to be specifically designed for execution of one or more of the operations of method 400.

At an operation 402, a reference set of record attributes are used to predict matching records in a first portion of a collection of healthcare records. At operation 402, the reference set of record attributes is used to process the first portion of the collection of healthcare records of individuals to generate a first prediction of which healthcare records of the first collection portion have matching values with respect to the reference set of record attributes. The reference set of record attributes includes one or more reference record attributes. The reference set of record attributes comprises one or more record attributes or combination of record attributes that are known to be reliable for accurately predicting a match between two healthcare records when the known reliable attributes or combination of record attributes of the two healthcare records have respective matching values. In some embodiments, at least one of the other sets of record attributes includes no personally identifiable information attributes. The first prediction indicates a first set of matches between healthcare records of the first collection portion.

By way of a non-limiting example, operation 402 may include determining known reliable features and/or feature combinations for a first portion of patient healthcare records in a first database and matching, using at least one of the known reliable features and/or feature combinations, the first portion of patient healthcare records in the first database to a first portion of corresponding patient healthcare records in a second database that share the at least one known reliable feature and/or feature combination. In some embodiments, operation 402 is performed by a processor component the same as or similar to ground truth component 32 (shown in FIG. 1 and described herein).

At an operation 404, other sets of record attributes are used to predict matching records in the first portion of the collection of healthcare records. For each set of other sets of record attributes the other set of record attributes is used to process the first collection portion to generate a second prediction of which healthcare records of the first collection portion have matching values with respect to the other set of record attributes. Each of the other sets of record attributes includes one or more record attributes different from the one or more reference record attributes, and each of the second predictions indicates a second set of matches between healthcare records of the first collection portion. For example, the matching prediction operation may be rerun on the same data (e.g., the first portion of the collection of healthcare records) but with different sets of record attributes to determine whether the different sets of record attributes predict the same matches predicted by the reference (e.g., the known reliable) attributes. In some embodiments, operation 404 is performed by a processor component the same as or similar to testing component 34 (shown in FIG. 1 and described herein).

At an operation 406, statistical information regarding the use of the other sets of record attributes for predicting healthcare record matches is determined. The statistical information is determined based on the first set of matches and the second set of matches. In some embodiments, operation 406 may comprise testing the reliabilities of the other sets of record attributes as record matching predictors (e.g., do the other sets of record attributes predict the same matches predicted by the reference sets of attributes?). In some embodiments, the statistical information comprises F-scores for individual other sets of record attributes and includes information regarding one or more true positives, false positives, true negatives, or false negatives related to predicted matches. In some embodiments, operation 406 is performed by a processor component the same as or similar to testing component 34 (shown in FIG. 1 and described herein).

At an operation 408, at least one of the other sets of record attributes is selected (e.g., based on its determined reliability) over at least another one of the other sets of healthcare record attributes for use in predicting healthcare record matches. The selection is made based on the statistical information and/or other information. In some embodiments, operation 408 includes comparing F-scores for the individual other sets of record attributes and selecting, for use in predicting healthcare record matches, at least one of the other sets of record attributes based on the comparison. In some embodiments, operation 408 includes selecting, for use in predicting healthcare record matches, at least one of the other sets of record attributes based on the comparison indicating the selected other set of record attributes has an F-score greater than or equal to an F-score for at least another one of the other sets of record attributes. In some embodiments, operation 408 includes selecting, for use in predicting healthcare record matches, at least one of the other sets of record attributes based on the comparison indicating the selected other set of record attributes has an F-score that satisfies a reliability threshold. In some embodiments, operation 408 is performed by a processor component the same as or similar to selection component 36 (shown in FIG. 1 and described herein).

At an operation 410, one or more other portions of the collection of healthcare records of individuals are processed, using the selected other set of record attributes, to generate a third prediction of which healthcare records of the other collection portions have matching values with respect to the selected other set of record attributes. In some embodiments, operation 410 includes processing, using an F-score based higher ranked first selected other set of record attributes, a first other portion of the collection of healthcare records of individuals to generate the third prediction; and processing, using an F-score based next ranked second selected other set of record attributes, a second other portion of the collection of healthcare records of individuals to generate a fourth prediction of which healthcare records of the second other collection portion have matching values with respect to the next ranked second selected other set of record attributes.

In some embodiments, operation 410 includes iteratively using a highest ranked other set of record attributes, a next highest ranked other set of record attributes, and so on, to generate predictions of which healthcare records of the other collection portions have matching values. This iterative matching may continue until stopping criteria is satisfied. In some embodiments, the stopping criteria comprises one or more of predicting matches for a predetermined quantity of records, a lack of remaining other sets of record attributes whose F-scores breach a reliability threshold, a matched record probability threshold, and/or other criteria. In some embodiments, operation 410 includes adjusting the matching predictions based on an edit distance associated with values of the set of record attributes. In some embodiments, operation 410 is performed by a processor component the same as or similar to matching component 38 (shown in FIG. 1 and described herein).

Although the description provided above provides detail for the purpose of illustration based on what is currently considered to be the most practical and preferred embodiments, it is to be understood that such detail is solely for that purpose and that the disclosure is not limited to the expressly disclosed embodiments, but, on the contrary, is intended to cover modifications and equivalent arrangements that are within the spirit and scope of the appended claims. For example, it is to be understood that the present disclosure contemplates that, to the extent possible, one or more features of any embodiment can be combined with one or more features of any other embodiment.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

1. A system configured to facilitate computer-assisted linkage of healthcare records, the system comprising one or more hardware processors configured by machine readable instructions to:
process, using a reference set of record attributes, a first portion of a collection of healthcare records of individuals to generate a first prediction of which healthcare records of the first collection portion have matching values with respect to the reference set of record attributes, the reference set of record attributes including one or more reference record attributes, and the first prediction indicating a first set of matches between healthcare records of the first collection portion;
for each set of other sets of record attributes:
process, using the other set of record attributes, the first collection portion to generate a second prediction of which healthcare records of the first collection portion have matching values with respect to the other set of record attributes, each of the other sets of record attributes including one or more record attributes different from the one or more reference record attributes, and each of the second predictions indicating a second set of matches between healthcare records of the first collection portion; and
determine, based on the first set of matches and the second set of matches, statistical information regarding use of the other set of record attributes for predicting healthcare record matches;
select, based on the statistical information regarding use of one or more of the other sets of record attributes, at least one of the other sets of record attributes over at least another one of the other sets of healthcare record attributes for use in predicting healthcare record matches; and
process, using the selected other set of record attributes, one or more other portions of the collection of healthcare records of individuals to generate a third prediction of which healthcare records of the other collection portions have matching values with respect to the selected other set of record attributes.

2. The system of claim 1, wherein the reference set of record attributes comprise one or more record attributes or combination of record attributes that are known to be reliable for accurately predicting a match between two healthcare records when the known reliable attributes or combination of record attributes of the two healthcare records have respective matching values, and wherein at least one of the other sets of record attributes includes no personally identifiable information attributes.

3. The system of claim 1, wherein the statistical information comprises F-scores for individual other sets of record attributes and includes information regarding one or more true positives, false positives, true negatives, or false negatives and wherein the one or more hardware processors are configured to:
compare F-scores for individual other sets of record attributes; and
select, for use in predicting healthcare record matches, at least one of the other sets of record attributes based on the comparison.

4. The system of claim 3, wherein the one or more hardware processors are configured to select, for use in predicting healthcare record matches, at least one of the other sets of record attributes based on the comparison indicating the selected other set of record attributes has an F-score greater than or equal to an F-score for at least another one of the other sets of record attributes.

5. The system of claim 3, wherein the one or more hardware processors are configured to select, for use in predicting healthcare record matches, at least one of the other sets of record attributes based on the comparison indicating the selected other set of record attributes has an F-score that satisfies a reliability threshold.

6. The system of claim 3, wherein the one or more hardware processors are configured to:
process, using an F-score based higher ranked first selected other set of record attributes, a first other portion of the collection of healthcare records of individuals to generate the third prediction; and
process, using an F-score based next ranked second selected other set of record attributes, a second other portion of the collection of healthcare records of individuals to generate a fourth prediction of which healthcare records of the second other collection portion have matching values with respect to the next ranked second selected other set of record attributes.

7. A method for facilitating computer-assisted linkage of healthcare records with a linkage system, the system comprising one or more hardware processors configured by machine readable instructions, the method comprising:
processing, using a reference set of record attributes, a first portion of a collection of healthcare records of individuals to generate a first prediction of which healthcare records of the first collection portion have matching values with respect to the reference set of record attributes, the reference set of record attributes including one or more reference record attributes, and the first prediction indicating a first set of matches between healthcare records of the first collection portion;
for each set of other sets of record attributes:
processing, using the other set of record attributes, the first collection portion to generate a second prediction of which healthcare records of the first collection portion have matching values with respect to the other set of record attributes, each of the other sets of record attributes including one or more record attributes different from the one or more reference record attributes, and each of the second predictions indicating a second set of matches between healthcare records of the first collection portion; and
determining, based on the first set of matches and the second set of matches, statistical information regarding use of the other set of record attributes for predicting healthcare record matches;
selecting, based on the statistical information regarding use of one or more of the other sets of record attributes, at least one of the other sets of record attributes over at least another one of the other sets of healthcare record attributes for use in predicting healthcare record matches; and
processing, using the selected other set of record attributes, one or more other portions of the collection of healthcare records of individuals to generate a third prediction of which healthcare records of the other collection portions have matching values with respect to the selected other set of record attributes.

8. The method of claim 7, wherein the reference set of record attributes comprise one or more record attributes or combination of record attributes that are known to be reliable for accurately predicting a match between two healthcare records when the known reliable attributes or combination of record attributes of the two healthcare records have respective matching values, and wherein at least one of the other sets of record attributes includes no personally identifiable information attributes.

9. The method of claim 7, wherein the statistical information comprises F-scores for individual other sets of record attributes and includes information regarding one or more true positives, false positives, true negatives, or false negatives and wherein the method further comprises:
comparing F-scores for individual other sets of record attributes; and
selecting, for use in predicting healthcare record matches, at least one of the other sets of record attributes based on the comparison.

10. The method of claim 9, further comprising selecting, for use in predicting healthcare record matches, at least one of the other sets of record attributes based on the comparison indicating the selected other set of record attributes has an F-score greater than or equal to an F-score for at least another one of the other sets of record attributes.

11. The method of claim 9, further comprising selecting, for use in predicting healthcare record matches, at least one of the other sets of record attributes based on the comparison indicating the selected other set of record attributes has an F-score that satisfies a reliability threshold.

12. The method of claim 9, further comprising:
processing, using an F-score based higher ranked first selected other set of record attributes, a first other portion of the collection of healthcare records of individuals to generate the third prediction; and
processing, using an F-score based next ranked second selected other set of record attributes, a second other portion of the collection of healthcare records of individuals to generate a fourth prediction of which healthcare records of the second other collection portion have matching values with respect to the next ranked second selected other set of record attributes.

13. A system configured to facilitate computer-assisted linkage of healthcare records, the system comprising means for:
processing, using a reference set of record attributes, a first portion of a collection of healthcare records of individuals to generate a first prediction of which healthcare records of the first collection portion have matching values with respect to the reference set of record attributes, the reference set of record attributes including one or more reference record attributes, and the first prediction indicating a first set of matches between healthcare records of the first collection portion;
for each set of other sets of record attributes:
processing, using the other set of record attributes, the first collection portion to generate a second prediction of which healthcare records of the first collection portion have matching values with respect to the other set of record attributes, each of the other sets of record attributes including one or more record attributes different from the one or more reference record attributes, and each of the second predictions indicating a second set of matches between healthcare records of the first collection portion; and
determining, based on the first set of matches and the second set of matches, statistical information regarding use of the other set of record attributes for predicting healthcare record matches;
selecting, based on the statistical information regarding use of one or more of the other sets of record attributes, at least one of the other sets of record attributes over at least another one of the other sets of healthcare record attributes for use in predicting healthcare record matches; and
processing, using the selected other set of record attributes, one or more other portions of the collection of healthcare records of individuals to generate a third prediction of which healthcare records of the other collection portions have matching values with respect to the selected other set of record attributes.

14. The system of claim 13, wherein the reference set of record attributes comprise one or more record attributes or combination of record attributes that are known to be reliable for accurately predicting a match between two healthcare records when the known reliable attributes or combination of record attributes of the two healthcare records have respective matching values, and wherein at least one of the other sets of record attributes includes no personally identifiable information attributes.

15. The system of claim 13, wherein the statistical information comprises F-scores for individual other sets of record attributes and includes information regarding one or more true positives, false positives, true negatives, or false negatives and wherein the means for processing, determining, and selecting are configured to:
compare F-scores for individual other sets of record attributes; and
select, for use in predicting healthcare record matches, at least one of the other sets of record attributes based on the comparison.

16. The system of claim 15, wherein the means for processing, determining, and selecting are configured to select, for use in predicting healthcare record matches, at least one of the other sets of record attributes based on the comparison indicating the selected other set of record attributes has an F-score greater than or equal to an F-score for at least another one of the other sets of record attributes.

17. The system of claim 15, wherein the means for processing, determining, and selecting are configured to select, for use in predicting healthcare record matches, at least one of the other sets of record attributes based on the comparison indicating the selected other set of record attributes has an F-score that satisfies a reliability threshold.

18. The system of claim 15, wherein the means for processing, determining, and selecting are configured to:
process, using an F-score based higher ranked first selected other set of record attributes, a first other portion of the collection of healthcare records of individuals to generate the third prediction; and
process, using an F-score based next ranked second selected other set of record attributes, a second other portion of the collection of healthcare records of individuals to generate a fourth prediction of which healthcare records of the second other collection portion have matching values with respect to the next ranked second selected other set of record attributes.
